(19) United States
(12) Patent Application Publication
(10) Pub. No.: US 2026/0248820 A1
(43) Pub. Date: Aug. 27, 2026

(54) USES OF HYDROGENATED CANNABIDIOL (H4CBD) AND ADVANCED METABOLIC SYNDROME

(71) Applicant: The Regents of the University of California, Oakland, CA (US)

(72) Inventors: Rudy M. ORTIZ, Merced, CA (US); Jessica WILSON, Merced, CA (US); Mark MASCAL, Merced, CA (US); Nikolay SHEVCHENKO, Merced, CA (US)

(73) Assignee: The Regents of the University of California, Oakland, CA (US)

(21) Appl. No.: 18/875,846

(22) PCT Filed: Jun. 29, 2023
(86) PCT No.: PCT/US2023/069356

§ 371 Date: Dec. 17, 2024

Related U.S. Application Data
(60) Provisional application No. 63/356,913, filed on Jun. 29, 2022.

(51) Int. Cl. A61K 31/00; A61P 3/04

(52) U.S. Cl. CPC A61K 31/658; A61P 3/04

(57) ABSTRACT

Aspects of the present disclosure relate to the use of the synthetic, hydrogenated CBD (H4CBD) for treating metabolic dysfunction and associated conditions.

### CROSS REFERENCE TO RELATED APPLICATIONS

This application claims priority to U.S. Provisional Patent Application Ser. No. 63/356,913, filed Jun. 29, 2022, which is incorporated by reference herein in its entirety.

### SEQUENCE LISTING

The instant application contains a Sequence Listing which has been submitted in XML format and is hereby incorporated by reference in its entirety. Said XML copy, created on Jun. 29, 2023, is named “UCMDP0002WO.XML” and is 2,819 bytes in size.

### BACKGROUND

### I. Field of the Invention

This invention relates generally to the fields of pharmacology and medicine.

### II. Background

The popularity of cannabis use in older adults in the United States has more than doubled over the past two decades. Sentiments toward cannabis as a viable treatment intervention continues to be well documented as a low risk alternative to pharmaceuticals. Among the age-related ailments for which medicinal cannabis is sought, chronic pain, irritable bowel syndrome, and glaucoma are the most frequently cited. However, a common consequence of aging is impaired substrate metabolism, which can result in the onset of metabolic conditions ranging in severity from risk factors for metabolic syndrome (MetS) to frank Type II diabetes mellitus (T2DM). The prevalence of MetS is 54.9% among people ≥60 years of age and the average age at diagnosis of T2DM is 46 with equal abundance across racial and ethnic groups. Importantly, the effects of herbal cannabis and cannabis constituent use during conditions of metabolic dysfunction are vastly understudied and the explosion of interest in cannabis amongst older adults, coupled with the relatively high incidence of MetS in this population, constitutes a critical intersection between cannabis users and advanced metabolic dysfunction.

Cannabidiol (CBD) is an abundant, non-intoxicating constituent of Cannabis sativa, which is of particular interest for pharmacological investigation. The legislative ambiguity and increasing ease-of-access to unregulated cannabis constituents have prompted endeavors to synthesize analogues of natural cannabinoids. The use of these synthetic cannabinoids circumvents the complex regulatory landscape surrounding the production of scheduled compounds, while at the same time providing a chemically-pure compound free from THC, primary psychoactive cannabinoid extracted from the cannabis, or pesticides. Synthetic, hydrogenated CBDs are non-intoxicating and offer similar therapeutic effects to that of natural CBD. For example, dihydrocannabidiol (H2CBD) was found to reduce PTZ-induced seizure frequency and severity in rats with equal effectiveness to that of CBD1. H4CBD is a compound that differs from CBD by the saturation of the two double bonds in the terpene fragment of the molecule. Like natural CBD, H4CBD has little affinity for the endocannabinoid receptors responsible for cannabis intoxication. Although its use in vivo has not been previously described, it is expected to exert effects similar to those of natural CBD.

Rigorous pre-clinical data are sparse concerning the effects of CBD in models of metabolic dysfunction. Natural CBD reduced the incidence of diabetes in diet-induced obese (DIO) and non-obese diabetic mice^{2,3}, providing some evidence that synthetic H4CBD has the potential to similarly ameliorate dysregulated metabolism. As the prevalence of MetS continues to increase in the US and globally, identifying novel compounds to ameliorate the multiple morbidities that characterize MetS becomes increasingly more important. Addressing the many maladies that comprise MetS simultaneously is challenging, which contributes to the urgency to develop novel therapies. Therefore, there exists a need for therapeutics for treating MetS and associated conditions.

### SUMMARY

Disclosed herein are methods and compositions for treating metabolic dysfunction and conditions associated with metabolic dysfunction. In some aspects, a method of treating metabolic dysfunction or a condition associated with metabolic dysfunction comprises administering a therapeutically effective amount of a composition comprising H4CBD to an individual in need thereof. In some aspects, the condition associated with metabolic dysfunction is selected from obesity, insulin resistance, reduced glucose tolerance, and metabolic syndrome. In some aspects, the individual has been diagnosed with metabolic dysfunction or a condition associated with metabolic dysfunction. In some aspects, the composition decreases body mass.

Some aspects of the disclosure are directed to a method of reducing body mass in an individual comprising administering to the individual a therapeutically effective amount of a composition comprising H4CBD. In some aspects, the reduced body mass comprises a reduction in abdominal fat.

In some aspects, the decrease in body mass is a reduction in abdominal fat. In some aspects, the composition increases insulin receptor expression. In some aspects, the composition reduces circulating adiponectin. In some aspects, the composition increases circulating ghrelin. In some aspects, the composition reduces plasma triglycerides. In some aspects, the composition increases lipid metabolism.

In some aspects, a method comprising administering a therapeutically effective amount of a composition comprising H4CBD to an individual increases insulin receptor expression. In some aspects, a method comprising administering a therapeutically effective amount of a composition comprising H4CBD to an individual reduces circulating adiponectin. In some aspects, a method comprising administering a therapeutically effective amount of a composition comprising H4CBD to an individual increases circulating ghrelin. In some aspects, a method comprising administering a therapeutically effective amount of a composition comprising H4CBD to an individual reduces plasma triglycerides. In some aspects, a method comprising administering a therapeutically effective amount of a composition comprising H4CBD to an individual increases lipid metabolism.

In some aspects, the H4CBD is a pharmaceutically acceptable salt, enantiomer, diastereomer, or prodrug thereof. In some aspects, the composition is administered orally, intraadiposally, intraarterially, intraarticularly, intracranially, intradermally, intralesionally, intramuscularly, intraperitoneally, intrapleurally, intranasally, intraocularally, intrapericardially, intraprostaticaly, intrarectally, intrathecally, intratumorally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof. In some aspects, the composition is administered to the subject at least two, three, four, five, six, seven, eight, nine or ten times. In some aspects, the H4CBD is administered at a dosage of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, or 20 mg/kg.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Bioavailability of drug in end of study plasma. Mean (±SEM) peak area count AUC relative abundance of H4CBD in 45-week-old Long Evan's Tokushima Otsuka (LETO) rat plasma (n=3), Otsuka Long-Evans Tokushima Fatty (OLETF) rat plasma (n=3) and H4CBD-treated OLETF rat plasma (H4CBD; n=6).

FIGS. 2A-2E H4CBD ameliorated glucose response but not hyperglycemia in advanced MetS. Mean (±SEM) FIG. 2A blood glucose % baseline response to Oral Glucose Tolerance Test (OGTT) and FIG. 2B corresponding AUC (r.u.), FIG. 2C insulin response % baseline to OGTT and FIG. 2D corresponding AUC (r.u.), FIG. 2E insulin resistance index (IRI; relative units) in 45-week-old LETO (n=8), OLETF (n=8) and H4CBD-treated OLETF (H4CBD; n=8). *p<0.01 LETO vs. OLETF, ‡p<0.01 LETO vs. H4CBD and ¥p<0.01 OLETF vs. H4CBD by 2-way ANOVA with Tukey's HSD. [symbol] p<0.05 LETO vs. OLETF, #p<0.05 LETO vs. H4CBD, \hat{ }p<0.05 OLETF vs. H4CBD by one-way ANOVA with Tukey's HSD. ¤p<0.05 OLETF vs. H4CBD by unpaired, one-tail ed t-test.

FIGS. 3A-3N H4CBD induced compensatory increase in skeletal muscle IR expression. FIG. 3A pIR, FIG. 3B IR, FIG. 3C pIR/IR, FIG. 3D pAkt, FIG. 3E Akt, FIG. 3F pAkt/Akt, FIG. 3G PI3K, FIG. 3H representative blots, FIG. 3I pAMPK, FIG. 3J AMPK, FIG. 3K pAMPK/AMPK, FIG. 3L membrane-bound GLUT4, FIG. 3M cytoplasmic GLUT4 and FIG. 3N Mem/cyto GLUT4 soleus protein expression in 41-45-week-old Long-Evans Tokushima Otsuka (LETO; n=8), Otsuka Long-Evans Tokushima Fatty (OLETF; n=8) and OLETF+H4CBD (H4CBD; n=8). [symbol] p<0.05 LETO vs. OLETF, #p<0.01 LETO vs. H4CBD LETO vs. H4CBD, \hat{ }p<0.05 OLETF vs. H4CBD by one-way ANOVA with Tukey's HSD.

FIGS. 4A-4E H4CBD reduced circulating adipokines but increased hunger hormone ghrelin in advanced MetS. Mean (±SEM) FIG. 4A plasma leptin, FIG. 4B plasma adiponectin, FIG. 4C plasma leptin: adiponectin, FIG. 4D plasma corticosterone and FIG. 4E plasma ghrelin in 45-week-old LETO (n=8), OLETF (n=8) and H4CBD-treated OLETF (H4CBD; n=8). #p<0.05 LETO vs. H4CBD and \hat{ }p<0.05 OLETF vs. H4CBD by one-way ANOVA with Tukey's HSD.

FIG. 5 H4CBD-induced reductions of abdominal fat and plasma adiponectin are positively correlated. Pearson r correlation values of end of study leptin, adiponectin, food intake (FI), epidydimal (epi.) fat, retroperitonal (retro.) fat, total (epi.+retro.) fat, leptin: adiponectin in LETO (n=8), OLETF (n=8) and H4CBD (n=8).

FIGS. 6A-6E Hydrogenated cannabidiol (H4CBD) decreased body mass and increased activity. Mean (±SEM) FIG. 6A cumulative sum gain of body mass, FIG. 6B daily body mass, FIG. 6C relative food intake and FIG. 6D water intake in 41-45-week-old Long-Evans Tokushima Otsuka (LETO; n=8), Otsuka Long-Evans Tokushima Fatty (OLETF; n=8) and OLETF+H4CBD (H4CBD; n=8). FIG. 6E Activity score [DSI; HD-S10] of OLETF (OLETF; n=2) and OLETF+H4CBD (H4CBD; n=3). *p<0.05 LETO vs. OLETF, &p<0.01 LETO vs. H4CBD, % p<0.05 OLETF vs. H4CBD 2-way ANOVA with Tukey's HSD.

FIGS. 7A-7E Synthetic CBD did not promote muscle wasting in advanced MetS. Mean (±SEM) FIG. 7A daily urine excretion (mL) (n=8 all groups), FIG. 7B total urinary protein excretion, FIG. 7C total urinary creatinine (Cr) excretion, FIG. 7D total urinary 3-methylhistidine (3MH) excretion and FIG. 7E 3MH/Cr ratio in 45-week-old LETO (n=5), OLETF (n=5) and H4CBD-treated OLETF (H4CBD; n=5). *p<0.05 LETO vs. OLETF, [symbol] p<0.01 LETO vs. H4CBD 2-way ANOVA with Tukey's HSD. ‡p<0.05 LETO vs. OLETF, #p<0.05 LETO vs. H4CBD, \hat{ }p<0.05 OLETF vs. H4CBD by 1-way ANOVA with Tukey's HSD. § p<0.05 LETO Day 0 vs. LETO Day 28, [symbol] p<0.01 OLETF Day 0 vs OLETF Day 28.

FIGS. 8A-8G Synthetic CBD reduced visceral fat mass in advanced MetS. Mean (±SEM) FIG. 8A relative retroperitoneal fat mass, FIG. 8B relative epidydimal fat mass and FIG. 8C combined visceral adipose. Representative images of adipocytes from retroperitoneal adipose. FIG. 8D LETO, FIG. 8E OLETF, FIG. 8F H4CBD. Mean (±SEM) FIG. 8G adipocyte size distribution in 45-week-old LETO (n=8), OLETF (n=8) and H4CBD-treated OLETF (H4CBD; n=8). [symbol] p<0.0001 LETO vs. OLETF, #p<0.05 LETO vs. H4CBD, \hat{ }p<0.05 OLETF vs. H4CBD by 1-way ANOVA with Tukey's HSD.

FIGS. 9A-9E H4CBD ablated dyslipidemia. Mean (±SEM) FIG. 9A adipose lipase activity, FIG. 9B liver cytosol lipase activity and FIG. 9C liver membrane lipase activity, FIG. 9D plasma triglycerides, FIG. 9E plasma non-esterified fatty acids (NEFA) and (F) plasma lipase activity in 45-week-old LETO (n=8), OLETF (n=8) and H4CBD-treated OLETF (H4CBD; n=8). [symbol] p<0.0001 LETO vs. OLETF, #p<0.05 LETO vs. H4CBD, \hat{ }p<0.05 OLETF vs. H4CBD by 1-way ANOVA with Tukey's HSD. ¥p<0.05 LETO vs. OLETF one-tailed unpaired t-test.

FIGS. 10A-10D H4CBD increased CD36 and FATP2 expression but not fat storage. Mean (±SEM) hepatic FIG. 10A FATP5 FIG. 10B FATP2, FIG. 10C CD36 and FIG. 10D triglyceride content in 45-week-old LETO (n=5-8), OLETF (n=5-8) and H4CBD-treated OLETF (H4CBD; n=6-7). [symbol] p<0.01 LETO vs. OLETF, \hat{ }p<0.05 OLETF vs H4CBD and #p<0.01 LETO vs. H4CBD by one-way ANOVA w/Tukey's HSD. § p<0.01 LETO vs. OLETF, *p<0.05 LETO vs. H4CBD and ‡p<0.05 OLETF vs H4CBD by one-tailed unpaired t-test.

FIG. 11 H4CBD did not contribute to liver injury. Collagen Type IV in 45-week-old LETO (n=5), OLETF (n=5) and H4CBD-treated OLETF (H4CBD; n=6). [symbol] p<0.01 LETO vs. OLETF and #p<0.01 LETO vs. H4CBD by one-way ANOVA w/Tukey's HSD.

FIGS. 12A-12F Synthetic CBD (H4CBD) does not reduce SBP or modulate hypertension drivers. (±SEM) FIG. 12A systolic blood pressure (SBP; mmHg) of OLETF (n=2) and OLETF+H4CBD (n=3) rats >41 weeks of age over 4 weeks. Mean (±SEM) end of study plasma FIG. 12B angiotensin II and FIG. 12C angiotensin 1-7, FIG. 12D aldosterone in LETO (n=7), OLETF control (n=6-8) and H4CBD-treated OLETF (n=7-8). Cardiac mRNA relative expression of FIG. 12E At1 and FIG. 12F Mas1 in LETO (n=6-8), OLETF control (n=6-7) and H4CBD-treated OLETF (n=6-7). *p<0.01 LETO vs. OLETF, \hat{ }p<0.05 OLETF vs. H4CBD, #p<0.01 LETO vs. H4CBD 1-way ANOVA with Tukey's HSD. ¥p<0.01 LETO vs. OLETF student's unpaired t-test.

### DETAILED DESCRIPTION

The effects of the synthetic, non-narcotic cannabinoid 1,2,8,9-tetrahydrocannabidiol (H4CBD) were examined due to its synthetic accessibility in pure form and potential for greater adoption than CBD, which is subject to multiple regulatory restrictions worldwide. Specifically, the effect of H4CBD on MetS cluster factors such as glucose intolerance and insulin resistance during advanced metabolic dysfunction were examined. Because of the explosion of interest in cannabis and cannabis constituents in older adults and the high incidence of MetS and cardiovascular disease of this population, a critical intersection of cannabis constituent use and advanced metabolic dysfunction exists. The Otsuka Long-Evans Tokushima Fatty (OLETF) rat is a monogenic model of diet-induced obesity accelerated by a mutation in the CCK receptor. These rats have a predictable, timed progression toward non-insulin dependent diabetes mellitus (T2DM) (>20 weeks), marked by a linear phase progression of hypertension (8-20 weeks), which closely resembles symptoms displayed by human T2DM symptoms including visceral adiposity, dyslipidemia and insulin resistance. At >40 weeks of age, OLETF rats suffer from severe metabolic dysfunction and therefore serve as a model of aged, severe MetS.

The meaning of certain terms as intended is defined herein below.

### I. Definitions

As used herein, the terms “H4CBD,” “1,2,8,9-tetrahydrocannabidiol,” “hydrogenated cannabidiol,” and “synthetic cannabidiol” refer to 2-(2-isopropyl-5-methylcyclohexyl)-5-pentylbenzene-1,3-diol. “Metabolic syndrome” is a cluster of a cluster of biochemical and physiological abnormalities associated with the development of cardiovascular disease and type 2 diabetes.

“Individual, “subject,” and “patient” are used interchangeably and can refer to a human or non-human.

The terms “lower,” “reduced,” “reduction,” “decrease,” or “inhibit” are all used herein generally to mean a decrease by a statistically significant amount. However, for avoidance of doubt, “lower,” “reduced,” “reduction, “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

Suitable pharmaceutically acceptable salts may also be formed by reacting the agents of the invention with an organic base such as methylamine, ethylamine, ethanolamine, lysine, ornithine and the like. Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable.

Additional examples of pharmaceutically acceptable salts and their methods of preparation and use are presented in Handbook of Pharmaceutical Salts: Properties, Selection and Use (2002), which is incorporated herein by reference.

### II. Pharmaceutical Compositions

The present disclosure includes methods for treating metabolic dysfunction or conditions associated with metabolic dysfunction.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2-day to 12-week intervals, more usually from one to two week intervals.

In some embodiments, a pharmaceutical composition comprises a pharmaceutically acceptable carrier or excipient. The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

### III. Administration of Therapeutic Compositions

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, the composition is administered at a dose of between 1 mg/kg and 5000 mg/kg. In some embodiments, the composition is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Effect of H4CBD on Glucose Tolerance and Insulin Resistance

Animals—The Otsuka Long-Evans Tokushima Fatty (OLETF) rat is a monogenic model of diet-induced obesity accelerated by a mutation in the CCK receptor. These rats have a predictable, timed progression toward non-insulin dependent diabetes mellitus (T2DM) (>20 weeks), marked by a linear phase progression of hypertension (8-20 weeks), which closely resembles symptoms displayed by human T2DM symptoms including visceral adiposity, dyslipidemia, and insulin resistance. At >40 weeks of age, OLETF rats suffer from severe metabolic dysfunction and therefore serve as a model of aged, severe MetS.

Male lean Long Evan's Tokushima Otsuka (LETO) rats and obese Otsuka Long Evans Tokushima Fatty (OLETF) rats (Japan SLC Inc., Hamamatsu, Japan), both 14 weeks of age were assigned to the following groups (n=8/group): (1) vehicle-dosed LETO (LETO), (2) vehicle-dosed OLETF (OLETF), and (3) H4CBD-treated (200 mg/kg/day×4 weeks) OLETF (H4CBD). Rats were maintained in a specific pathogen-free, climate-controlled facility on a 12-hour light: dark cycle (07:00-19:00 and 19:00-07:00, respectively). All animals had free access to water and were fed rat chow (Teklad Global; fat 9.0%; carbohydrate 44.9%; protein 19.0%) ad libitum. Treatment intervention was initiated at 41 weeks of age. Phenotypic data (i.e. body mass, food intake) was collected daily for all animals.

Drug Preparation and Administration—H4CBD (1,2,8,9-tetrahydrocannabidiol, systematic name 2-(2-isopropyl-5-methylcyclohexyl)-5-pentylbenzene-1,3-diol), was prepared by reduction of synthetic H2CBD (8,9-tetrahydrocannabidiol) with hydrogen and a Pd/C catalyst in acetic acid solvent, and was purified by distillation in vacuo. H2CBD was synthesized from olivetol and food-grade a-phellandrene according to the published procedure¹. All chemicals were purchased from Millipore Sigma and used as received.

Purified H4CBD (>99%) was suspended in food grade sesame oil and administered by oral gavage at a dose of 200 mg/kg. This dose has been shown to be similarly effective on seizure frequency and severity in rats compared to natural CBD, which gave reasonable cause for efficacy of H4CBD at the same dose, and is well below documented toxicity of CBD (>600 mg/kg) in rodents⁵.

Oral Glucose Tolerance Test (OGTT)—At approximately 45 weeks of age and following an overnight fast, oGTTs were performed as previously described. The positive incremental areas under the curve for glucose (AUCglucose) and insulin (AUCinsulin) were calculated by the trapezoidal method. Insulin resistance index (IRI) was calculated by AUCglucosex AUCinsulin/100, as previously described.

Tissue Collection—After the 4-week study and 3 days following the OGTT, animals were fasted overnight and tissues were collected the following morning.

Western Blot—Soleus was used to measure proteins involved in insulin signaling. Densitometry values were quantified using ImageJ software (NIH) and normalized by correcting for densitometry values of representative protein bands below 37 kDa stained with Ponceau S. Results are reported as expression (%) compared to LETO.

Drug Bioavailability Detection—H4CBD bioavailability in heparinized plasma was determined via HPLC and MS/MS. In brief, 100 μL heparinized rat plasma was diluted with 100 μL Mass Spec Gold human serum (Golden West Diagnostics, MSG3200). The samples were analyzed using a WATERS TQS-Micro. The first quadrupole was set to 319.16 m/z and the third quadrupole isolated fragments of 181.02 m/z. Any sample that had a peak area count of <1500 was considered negative for H4CBD.

Statistics—All values are represented as mean #standard error mean (SEM) unless otherwise indicated. Means were compared by one-way ANOVA followed by Tukey's honest significant difference or unpaired, one-tailed t-test to assess significant differences among groups. Means and regressions were considered significant at p<0.05. Outliers were detected by ROUT (Q=1.0%) and removed; however, it should be noted that this was necessary for only 12 occurrences. All statistical procedures, including Pearson r correlations, were performed using GraphPad Prism 9.3.1 (GraphPad Software, Inc., San Diego, CA, USA).

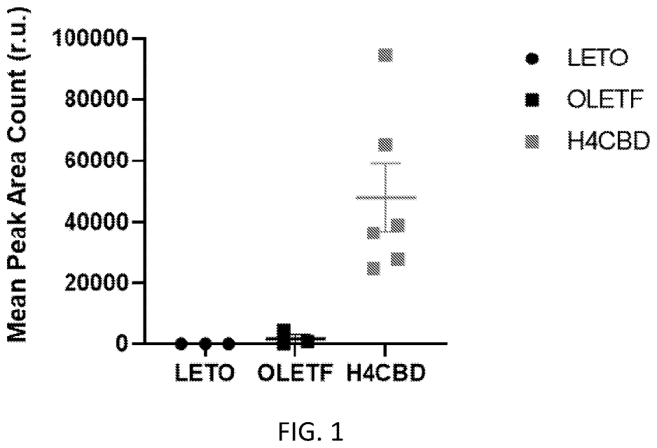

H4CBD was only detected in plasma from treated rats-Bioavailability of H4CBD compound was validated in end of study plasma and confirmed that only treated animals received drug (FIG. 1).

H4CBD reduced body mass (BM) independent of changes in food consumption-BM and food consumption were measured daily, and abdominal fat masses were weighed at dissection, to determine the effect of H4CBD treatment on phenotypic indicators of metabolic dysfunction. H4CBD reduced BM to vehicle-treated LETO levels (Table 1). BM of vehicle-treated OLETF was 16% higher than LETO (p<0.05) and 15% higher (p<0.05) than H4CBD-treated OLETF (Table 1). Relative retroperitoneal fat, but not relative epidydimal fat, was 267% more abundant in OLETF than LETO (p<0.0001) (Table 1). H4CBD reduced relative retroperitoneal fat by 24% (p<0.05) and relative epidydimal fat by 35% (p<0.05) compared to OLETF control (Table 1). Relative combined (total) adipose was 149% higher in OLETF compared to LETO (p<0.0001), which was reduced by 25% with H4CBD treatment (p<0.05 LETO vs. H4CBD) (Table 1). The lower BM in H4CBD-treated animals was not associated with a change in food consumption compared to OLETF (Table 1).

TABLE 1
Mean ± SEM morphometrical measurements
in LETO, OLETF, and H4CBD-treated OLETF male rats

| | LETO | OLETF | H4CBD |
|---|---|---|---|
| | (n = 8) | (n = 8) | (n = 8) |
| Body mass (g) | 527 ± 10 | 640 ± 38* | 516 ± 21\hat{ }^{#} |
| Relative retro. fat (g/100 g BM) | 2.4 ± 0.1 | 8.6 ± 0.6* | 6.6 ± 0.9^{#} |
| Relative epi. fat (g/100 g BM) | 1.8 ± 0.1 | 1.8 ± 0.2 | 1.2 ± 0.1\hat{ }^{#} |
| Relative total fat (g/100 g BM) | 4.2 ± 0.2 | 10.5 ± 0.7* | 7.8 ± 1.1^{#} |
| Initial, absolute food inake (g) | 25 ± 2 | 32 ± 1* | 34 ± 2^{#} |
| Final, absolute food intake (g) | 21 ± 1 | 37 ± 2* | 36 ± 2^{#} |
| Relative final food intake | 3.9 ± 0.1 | 6.4 ± 0.7* | 6.7 ± 0.4^{#} |
| (g/100 g BM) | | | |

epi. = epidydimal,
retro. = retroperitoneal,
BM = body mass, food intake (average of 3 days)
*p < 0.05 LETO vs. OLETF; \hat{ }p < 0.05 OLETF vs. H4CBD; ^{#}p < 0.05 LETO vs. H4CBD one-way ANOVA with Tukey's HSD.

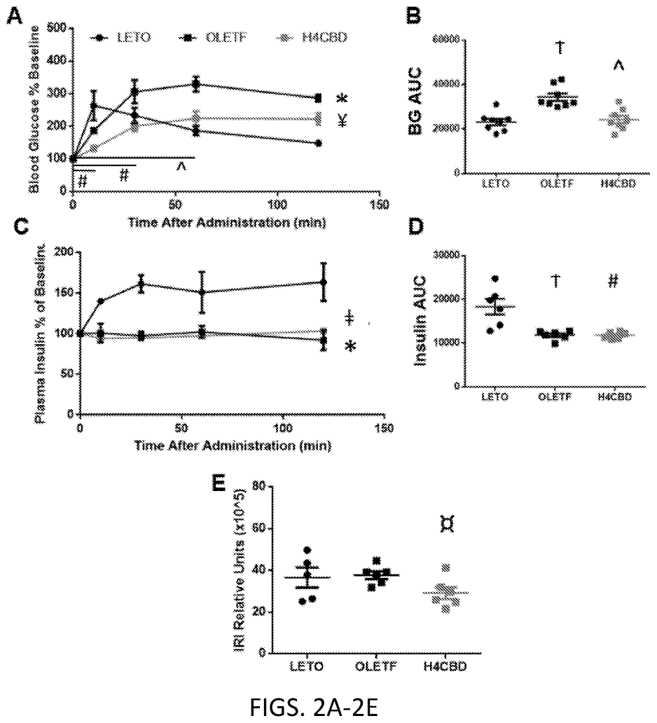

H4CBD ameliorated dynamic glucose response and insulin resistance index (IRI)—oGTTs were performed to determine the effects of H4CBD on glucose intolerance and degree of insulin resistance status during advanced MetS. LETO blood glucose response peaked at 10 minutes after glucose bolus (TO-T10 and TO-T30 AUCglucose p<0.05 LETO vs. H4CBD) (FIG. 2A). Treated and non-treated OLETF peaked 60 minutes after glucose bolus (TO-T60 AUCglucose p<0.01 OLETF vs. H4CBD) (FIG. 2A). Glucose response curve was lowest for H4CBD-treated animals overall (p<0.01 LETO vs. OLETF and OLETF vs. H4CBD) (FIG. 2A). Overall AUCglucose was 49% higher in OLETF compared to LETO (p<0.001) and H4CBD reduced AUCglucose 29% from OLETF (p<0.001) (FIG. 2B). Plasma insulin response was abolished in OLETF, which was not rescued by H4CBD treatment (FIGS. 2C-2D). IRI status was similar between aged LETO and OLETF rats, but IRI was reduced 23% in H4CBD (p<0.05 OLETF vs. H4CBD) (FIG. 2E).

H4CBD did not improve static indicators of glucose tolerance—At 45 weeks of age, fasting blood glucose (FBG) was 44% higher in OLETF than LETO while levels were 2.2-fold higher in H4CBD than LETO and 56% higher than OLETF (p<0.0001 LETO vs. H4CBD and p<0.01 OLETF vs. H4CBD) (Table 2). Fasting plasma insulin was 64% higher in OLETF compared to LETO (p<0.01) and H4CBD treatment increased levels by 10% over OLETF (p<0.001 LETO vs. H4CBD) (Table 2). Plasma glucagon was 63% lower in OLETF compared to LETO (p<0.05), but were not different from H4CBD (Table 2). H4CBD increased fasting glucose to insulin ratio by 47% compared to OLETF (p<0.05) (Table 2). H4CBD reduced fasting insulin to glucagon ratio by 64% (p<0.05) compared to OLETF (Table 2).

TABLE 2
Mean ± SEM end of study fasting plasma measurements

| | LETO | OLETF | H4CBD |
|---|---|---|---|
| | (n = 8) | (n = 8) | (n = 8) |
| Fasting blood glucose | 7.0 ± 0.4 | 10.1 ± 1.0 | 15.8 ± 1.9^{†£} |
| (mmol/L) | | | |
| Fasting insulin (μIU/mL) | 7.7 ± 0.7 | 12.6 ± 0.9* | 13.9 ± 0.4^{#} |
| Fasting glucagon (pg/mL) | 21.7 ± 5.0 | 8.0 ± 1.3* | 19.4 ± 9.4 |
| Glucose:Insulin (mmol/ | 8.7 ± 0.6 | 7.4 ± 0.7 | 10.9 ± 1.1\hat{ } |
| μIU × 10⁴) | | | |
| Insulin:Glucagon (μIU/pg) | 0.4 ± 0.0 | 1.6 ± 0.1* | 0.6 ± 0.0\hat{ } |

*p < 0.05 LETO vs. OLETF; \hat{ }p < 0.05 OLETF vs. H4CBD; ^{#}p < 0.05 LETO vs. H4CBD one-way ANOVA with Tukey's HSD; ^{†}p < 0.0001 LETO vs. H4CBD; ^{£}p < 0.01 OLETF vs. H4CBD unpaired one-tailed t-test.

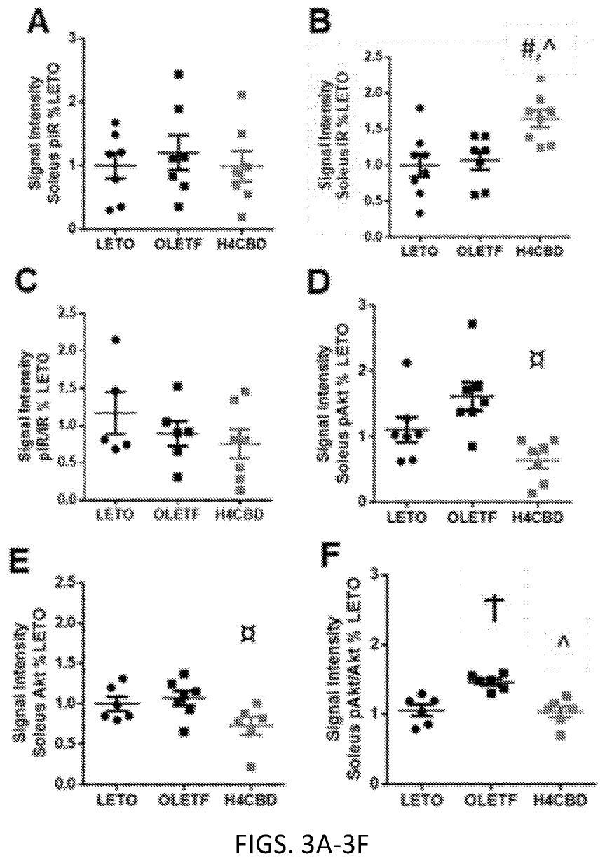

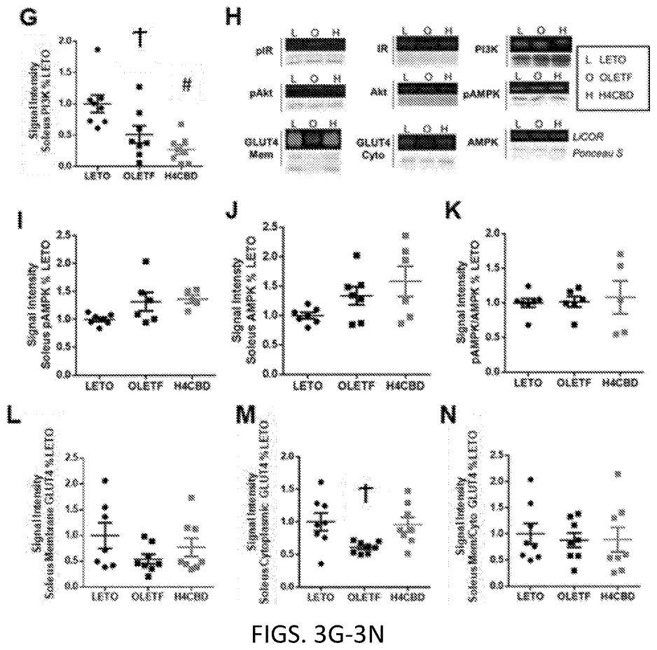

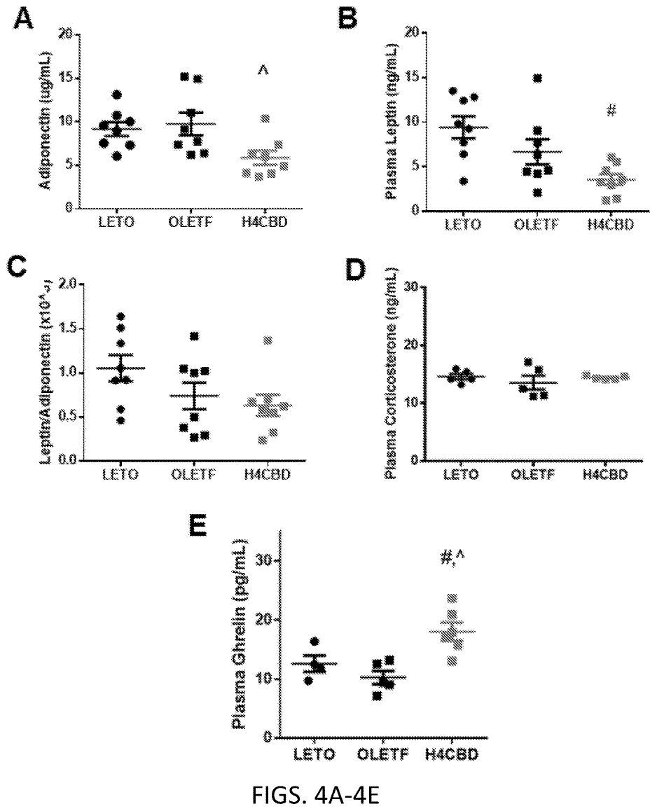

H4CBD statically increased insulin receptor expression—Skeletal muscle insulin signaling proteins were measured to assess the potential mechanisms by which H4CBD ameliorated the insulin resistance. No changes were detected in phosphorylation of the insulin receptor (pIR) (FIG. 3A). H4CBD increased native insulin receptor (IR) expression by 54% over OLETF (p<0.05 OLETF vs. H4CBD and p<0.01 LETO vs. H4CBD) (FIG. 3B); however, pIR/IR was not changed (FIG. 3C). Cytosolic pAkt and Akt expressions were comparable between LETO and OLETF, but H4CBD reduced the expressions of pAkt and Akt and the pAkt: Akt ratio by 58% (p<0.05), 32% (p<0.05), and 35% (p<0.01), respectively, compared to OLETF (FIGS. 3D-3F). PI3K expression was reduced 49% (p<0.05) in OLETF compared to LETO and H4CBD further reduced PI3K expression by 47% compared to OLETF (p=0.001 LETO vs. H4CBD) (FIG. 3G). No changes were detected in pAMPK, AMPK or pAMPK/AMPK expression (FIGS. 3I-3K).

Translocated GLUT4 may serve as an indicator of increased insulin signaling and was measured here by probing for its expression in the membrane fraction of skeletal muscle. However, there were no changes to translocated GLUT4 (FIG. 3L). Cytosolic GLUT4 was 39% lower in OLETF compared to LETO (p<0.05) and tended (p=0.06) to be higher (58%) in H4CBD compared to OLETF (FIG. 3M). The ratio of membrane to cytosolic GLUT4 was unchanged among the groups (FIG. 3N).

H4CBD reduced circulating adiponectin and leptin and increased ghrelin—Fasting plasma adiponectin, corticosterone, ghrelin, and leptin were measured to assess the effect of H4CBD on levels of adipocytokines and other hormones associated with insulin resistance and obesity. While there was no strain difference observed, H4CBD reduced circulating adiponectin by 40% (p<0.05) compared to OLETF (FIG. 3A). H4CBD reduced leptin by 47% compared to OLETF (p<0.05) but values were similar between LETO and OLETF (FIG. 3B). Although the leptin: adiponectin ratio was 30% lower in OLETF compared to LETO, H4CBD did not modulate the ratio further (FIG. 3C). Plasma corticosterone was comparable amongst the groups (FIG. 3D). Plasma ghrelin was 75% greater in H4CBD compared to the other groups and levels were similar between LETO and OLETF (p<0.01 OLETF vs. H4CBD and p<0.05 LETO vs. H4CBD) (FIG. 3E).

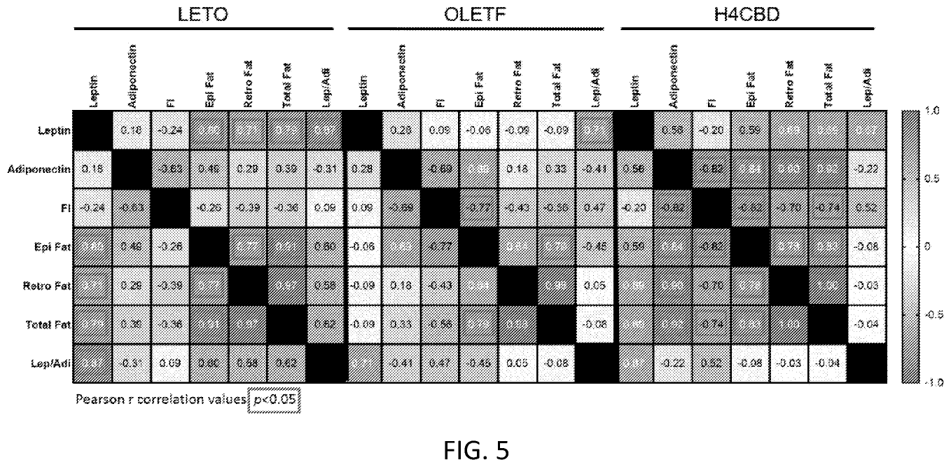

H4CBD-induced reduction of abdominal fat was positively correlated with adiponectin reduction—Pearson r correlations of end of study plasma leptin, adiponectin, food intake, epidydimal fat, retroperitoneal fat, total fat and leptin: adiponectin ratio were conducted to determine significant interactions between fat mass and hormones associated with insulin resistance and obesity. LETO abdominal fat was positively associated with plasma leptin (Pearson r 0.79; p<0.05) unlike OLETF (FIG. 5). H4CBD-induced reductions of abdominal fat was positively correlated with plasma adiponectin (Pearson r 0.92; p<0.01) and negatively correlated with food intake (Pearson r-0.74; p<0.05) (FIG. 5).

### DISCUSSION

The results disclosed herein indicate that in older OLETF rats (>40 weeks of age) fed ad libitum, fasting blood glucose (FBG) was 43% higher than LETO and that OLETF rats struggled to clear glucose from circulation long after time-to-clearance in LETO. Both metrics are consistent with previously characterized hyperglycemia in OLETF rats at 40 weeks of age⁴. Moreover, the insulin response to oGTT was severely blunted at this age, though fasting insulin remained relatively high in response to sustained hyperglycemia, which is indicative of (1) insufficient β-cell insulin secretion and (2) insulin resistance at the level of the receptor, respectively. Most importantly, treatment with H4CBD improved the strain-associated glucose intolerance independent of static enhancements in the insulin signaling pathway.

Interestingly, while H4CBD resulted in acute, dynamic improvements in glucose metabolism, the treatment did not appear to correct the chronically sustained hyperglycemia or hyperinsulinemia. The H4CBD-mediated improvement in glucose tolerance was modest, but biologically significant, and likely attributed, at least in part, to the reduction in adiposity.

### Example 2—Effect of H4CBD on Visceral Adiposity and Body Mass

The effects of H4CBD on MetS risk factors in advanced MetS were examined. Because of the explosion of interest in cannabis and cannabis constituents in older adults and the high incidence of MetS and CVD of this population, a critical intersection of cannabis constituent use and advanced metabolic dysfunction exists. The questions around the therapeutic benefits and risks of cannabis constituent consumption in conditions of severe metabolic dysfunction necessitate rigorous pre-clinical investigation. The inventors therefore examined the effects of H4CBD on parameters of lipid metabolism associated with MetS.

Animals—Male lean Long Evan's Tokushima Otsuka (LETO) rats and obese Otsuka Long Evans Tokushima Fatty (OLETF) rats (Japan SLC Inc., Hamamatsu, Japan), both 14 weeks of age were assigned to the following groups (n=8/group): (1) vehicle-dosed LETO (LETO), (2) vehicle-dosed OLETF (OLETF), and (3) H4CBD-treated (200 mg/kg/day×4 weeks) OLETF (H4CBD). Rats were maintained in a specific pathogen-free, climate-controlled facility at the University of California, Merced on a 12-hour light: dark cycle (07:00-19:00 and 19:00-07:00, respectively). All animals had free access to water and were fed rat chow (Teklad Global; fat 9.0%; carbohydrate 44.9%; protein 19.0%) ad libitum. Treatment intervention was initiated at 41 weeks of age.

Body Mass (BM), Water, and Food Intake—BM was measured daily to calculate the appropriate drug and vehicle dose. Water, urine and food were measured once every 24 hours throughout the study.

Telemeter Implantation—At 16 weeks, OLETF rats (n=5) were implanted with DSI telemeters (HD-S10) in the abdominal cavity to measure systolic blood pressure. LETO rats were not implanted; however, extensive telemetry data for these controls exist that demonstrates the maintenance of normotensive conditions including at a similar advanced age. Activity was measured every hour for 15 minutes for the duration of the study. Data were analyzed using A.R.T. software (DSI, St. Paul, MN).

Drug Preparation and Administration—Purified H4CBD (>99%) was suspended in sesame oil and administered by oral gavage at a dose of 200 mg/kg. This dose has been shown to be similarly effective on seizure frequency and severity in rats compared to natural CBD extract⁶, which gave reasonable cause for efficacy of H4CBD at the same dose, and is well below documented toxicity of CBD (>600 mg/kg) in rodents¹⁰.

Drug Bioavailability Detection—H4CBD bioavailability in heparinized plasma was determined via HPLC and MS chromatography.

Tissue Collection—After the 4-week study period, animals were fasted overnight, and tissues were collected at sacrifice the following morning. BM was recorded before animals were decapitated and trunk blood was collected.

Plasma Analysis—Plasma concentrations of adiponectin (Millipore Sigma; EZRADP-62K), aldosterone (R&D Systems; KGE016), angiotensin 1-7 (Phoenix Pharmaceuticals; EKE-002-01), angiotensin II (Phoenix Pharmaceuticals; EK-002-12CE), corticosterone (R&D Systems; KGE009), ghrelin (Sigma Aldrich; RAB0207), glucagon (Crystal Chemicals; 81519), leptin (Millipore Sigma; EZRL-83K), non-esterified fatty acid (NEFA) (Wako Chemicals) and triglycerides (Cayman Chemical; 10010303) were measured in fasted, end of study plasma samples. All samples were analyzed in duplicate and run in a single assay with intra-assay and percent CV of <10% for all assays.

Urinalysis—Urine was collected and thawed on ice to measure 3-methylhistidine (3MH; Abbexa; 257295) and creatinine (Invitrogen; EIACUN). Excretion was calculated for both by multiplying the 24-hr urine volume by the measured concentration (U×V=V*[x]), where x=creatinine or 3MH.

Western Blot—Liver was used to measure proteins involved in fatty acid uptake, metabolism and storage. Densitometry values were quantified using ImageJ software (NIH) and normalized by correcting for densitometry values of representative protein bands below 37 kDa stained with Ponceau S. Results are reported as expression (%) compared to LETO.

Lipase Activity Assay—White adipose tissue (WAT) and liver was assayed for lipase activity.

Real-Time qPCR—Gene expression was quantified by qPCR on cardiac tissue. Primers used were acquired from Integrated DNA Technologies and listed here 5′-3′: angiotensin MAS1 receptor (Mas1) Fw: AACACATGGGCCTCCCATTC (SEQ ID NO:1), Rv: AACAGGTAGAGGACCCGCAT (SEQ ID NO:2).

Statistics—All values are represented as mean±standard error mean (SEM) unless otherwise indicated. Means were compared by one-way ANOVA followed by Tukey's honest significant difference or unpaired, one-tailed t-test was to assess significant differences among groups. Means and regressions were considered significant at p<0.05. Outliers were detected by ROUT (Q=1.0%) and removed.

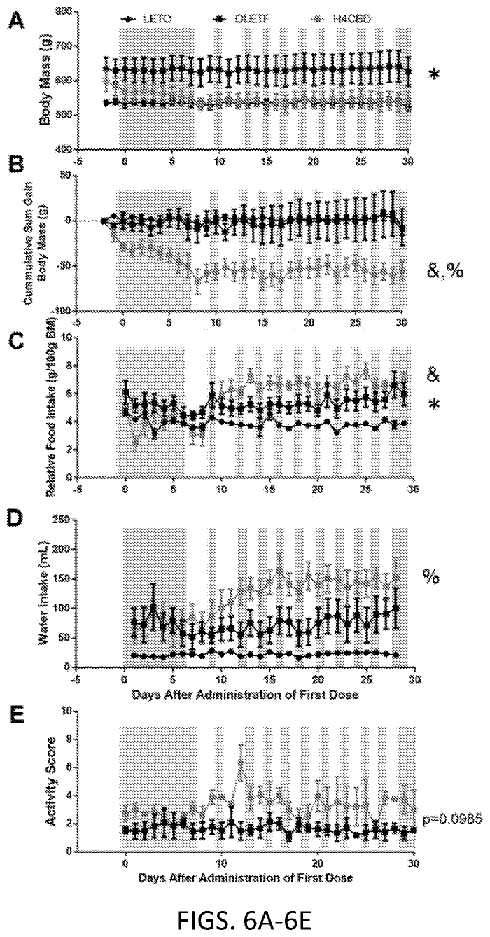

H4CBD reduced BM despite increase in food consumption—BM, relative food consumption, water consumption and activity were measured daily to determine the effect of CBD on phenotypic indicators of metabolic dysfunction. Bioavailability of drug compound was validated in end of study plasma and confirmed only treated animals received drug (Table 2). H4CBD reduced BM in OLETF to LETO levels within the first week and were maintained for the duration of the study (FIGS. 6A-6B). BM of untreated OLETF was 16% higher than LETO and 15% higher than H4CBD-treated OLETF after the first week of the study (day 10-30). BM reduction was in spite of an average 24% increased relative food consumption compared to OLETF control and 74% increased relative food intake consumption to LETO between day 10 and day 30 (FIG. 6C). Water intake was >200% higher in OLETF compared to LETO for the duration of the study (FIG. 6D). By day 13, H4CBD-treated OLETF water intake was 90% higher than OLETF control and >600% higher than LETO through the end of the study (FIG. 6D). Treated OLETF (H4CBD; n=3) displayed an increase in activity that was nearly double that of OLETF control, though this did not reach statistical significance (p=0.0985). FIG. 6E).

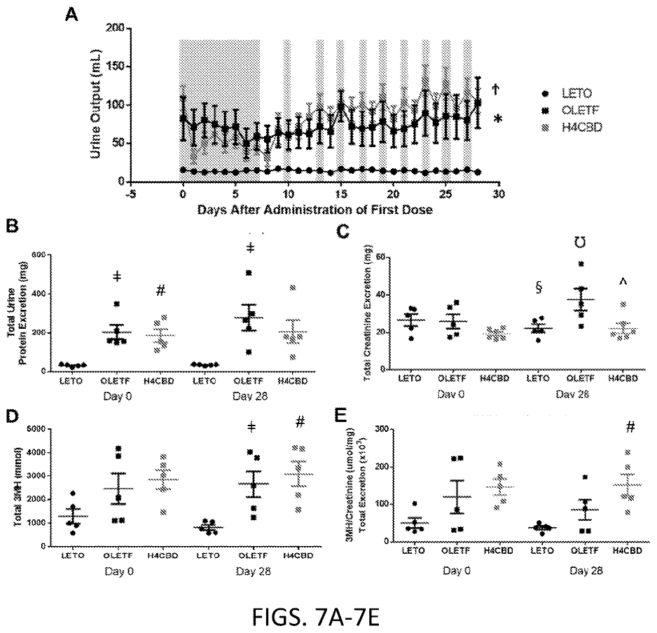

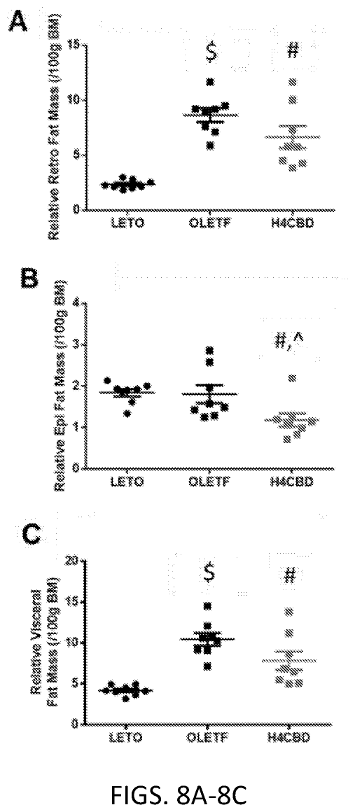

H4CBD did not promote muscle wasting—Excreted urine was measured and collected daily over 24-hour periods into an open vessel and analyzed for creatinine (Cr), 3-methylhistidine (3MH), and total protein content pre- and post-4-week H4CBD treatment to determine if H4CBD promoted muscle wasting via clinical 3MH/Cr ratio. Urine volume excretion was higher for OLETF compared to LETO (p<0.05) throughout the study (FIG. 7A). Although urine excretion was reduced in the first week for H4CBD-treated OLETFs compared to vehicle control, urine excretion volume increased above OLETF control by Day 11 and persisted through the remainder of the study (p<0.05 LETO vs. H4CBD) (FIG. 7A).

Total protein excretion pre-treatment (Day 0) was >500% higher in OLETF compared to LETO (p<0.01) (FIG. 7B). After 4 weeks, LETO total protein excretion did not significantly increase while OLETF tended to increase by 37% (p=0.053) (FIG. 7B). H4CBD treatment reduced proteinuria by 25% compared to OLETF control (FIG. 7B).

Creatinine excretion was statistically comparable on all groups prior to initiation of dose regimen (Day 0), though H4CBD pre-treatment group total creatinine excretion was 25% less (non-significant) than OLETF control (FIG. 7C). After 4 weeks, OLETF control creatinine levels increased by 45% (p<0.01), which widened the disparity between LETO and OLETF control groups to 65% (p<0.05) (FIG. 7C). Within H4CBD-treated group, creatinine excretion increased by a non-significant 10% after 4 weeks of treatment, which resulted in a 42% reduction compared to OLETF control overall (FIG. 7C).

3-methylhistidine (3MH) excretion is a product of amino acid breakdown, which is used clinically in ratio to urine creatinine to assess muscle wasting. Total 3MH excretion was not significantly different between all groups on Day 0 despite measuring 92% higher on average in OLETF compared to LETO on Day 0 (FIG. 7D). After 4 weeks, 3MH excretion non-significantly decreased by 37% in LETO and 8% in OLETF control between Day 0 and Day 28 (FIG. 7D). H4CBD-treated OLETF 3MH excretion increased by 9% pre-vs. post-treatment and was 17% less than OLETF control overall (FIG. 7D).

Pre-treatment 3MH/Cr ratio, therefore, was 138% higher in OLETF compared to LETO and 22% higher in the H4CBD-treated group compared to OLETF control; none of which reached statistical significance (FIG. 7E). After 4 weeks, LETO 3MH/Cr deceased by 25%, OLETF control decreased by 29% and H4CBD treated OLETF increased by 4% (FIG. 7E). Post-treatment (Day 28), OLETF 3MH/Cr was 128% higher than LETO (p=0.0597) and H4CBD-treated OLETF 3MH/Cr was 77% higher than OLETF control (p=0.07 OLETF vs. H4CBD, p<0.05 LETO vs. H4CBD) (FIG. 7E).

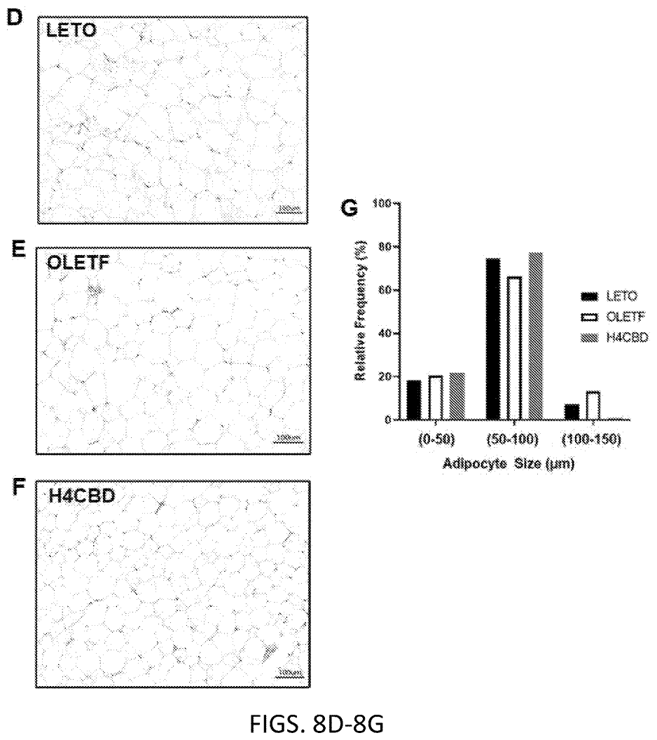

H4CBD reduced adiposity and adipocyte morphology—Visceral fat masses (retroperitoneal and epidydimal) were dissected and quantified to determine the effect of H4CBD on abdominal adiposity. Fasted plasma lipase activity, as well as plasma NEFA and TG, were measured to assess the effect of synthetic CBD on lipid metabolism parameters. Relative retroperitoneal fat, but not relative epidydimal fat, was 267% more abundant in OLETF than LETO (p<0.0001) (FIGS. 8A-8B). H4CBD reduced relative retroperitoneal fat by 24% (p<0.05) and relative epidydimal fat by 35% (p<0.05) compared to OLETF control (FIGS. 8A-8B). Relative combined adipose was 149% higher in OLETF compared to LETO (p<0.0001), which was reduced by 25% in OLETF with H4CBD treatment (p<0.05 LETO vs. H4CBD) (FIG. 8C).

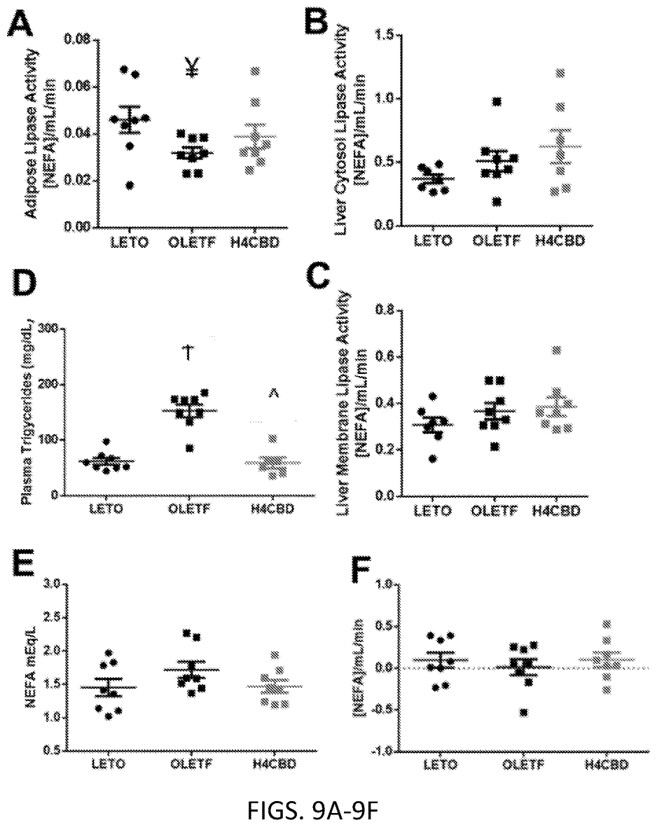

H4CBD ablated plasma triglycerides—Liver and adipose lipase activity was measured to determine the effect of H4CBD on the abundance of active lipases. The reduction in visceral adiposity and adipocyte size suggests that H4CBD activated and enhanced lipid metabolism as an alternative substrate metabolism. While fasted plasma lipase activity was not significantly different among groups (data not shown), adipose lipase activity was 31% (p<0.05) lower in OLETF compared to LETO (FIG. 9A). H4CBD tended to rescue adipose lipase activity with an average increase of 22% (p=0.094) compared to OLETF control (FIG. 9A). Liver endothelial membrane-bound lipases are known to contribute to TG breakdown in the blood stream. However, neither cytosolic nor liver lipase activity was not different among groups (FIGS. 9B-9C). Fasted plasma TG, the substrate of lipolysis, was 147% higher in OLETF compared to LETO (p<0.0001) and H4CBD treatment reduced fasted plasma TG to LETO control levels (p<0.0001 OLETF vs. H4CBD) (FIG. 9D). Fasted plasma NEFA and plasma lipase activity was not different among groups (FIGS. 9E-9F), which suggests a shift toward lipid metabolism.

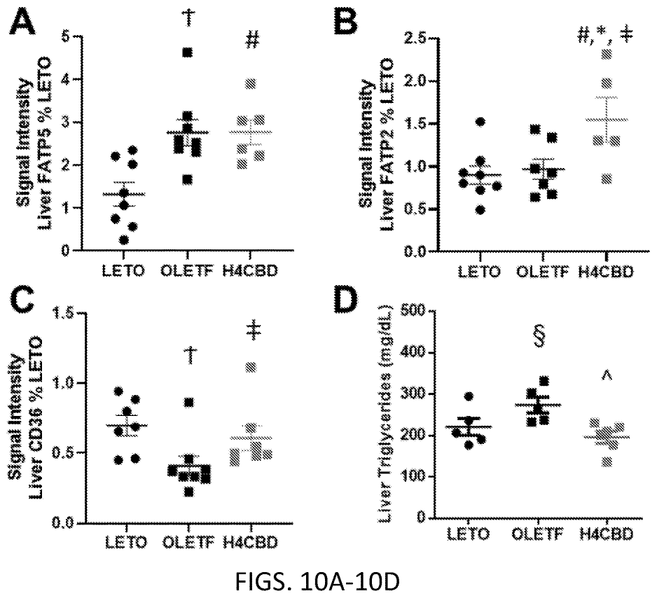

H4CBD promoted lipid metabolism via increased hepatic CD36 and FATP2 expression—Hepatic lipid metabolism signaling proteins were measured to assess the impact of H4CBD on lipid metabolism. FATP5 expression was increased 109% (p<0.01) in OLETF compared to LETO and was not effected by H4CBD treatment (FIG. 10A). FATP2 expression was comparable between LETO and OLETF but increased by 60% (p<0.05) in the H4CBD treated group (FIG. 10B). CD36 expression was 41% (p<0.05) lower in OLETF compared to LETO and H4CBD treatment rescued CD36 expression by 48% (p<0.05) (FIG. 10C). Downstream signaling proteins, GPAM, DGAT1, CPTIA, ACOX1, ApoB and PRDX6 were comparable between groups (data not shown). Liver triglyceride content was 24% (p<0.05) higher in OLETF compared to LETO and reduced in H4CBD-treated animals 28% (p<0.05) (FIG. 10D). Taken together, the increase of fatty acid transporter expression suggests H4CBD treatment enhanced lipid uptake to promote lipid metabolism.

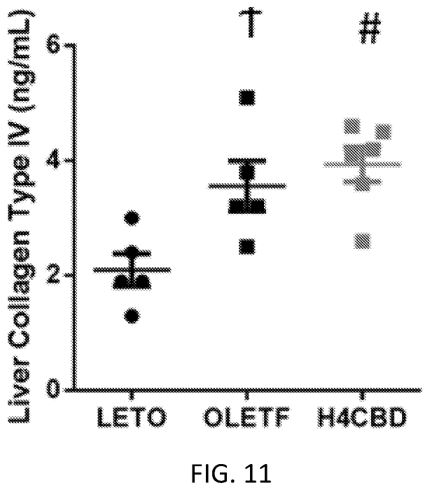

H4CBD did not contribute to liver damage—Indicators of damage were measured in liver tissue to assess the effects of H4CBD on hepatotoxicity. Type IV collagen deposition is an indicator of liver fibrosis, which is useful in the diagnosis of NAFLD in elderly individuals and models of NAFLD like OLETF. Liver collagen deposition was 71% (p<0.05) higher in OLETF compared to LETO and H4CBD treatment had no effect on collagen levels (FIG. 11).

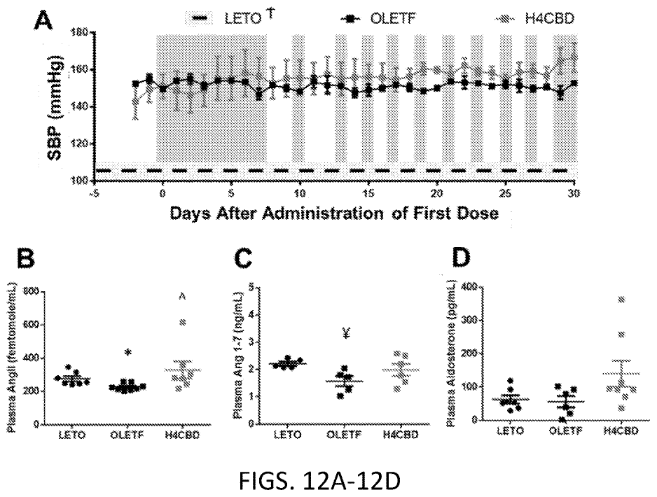

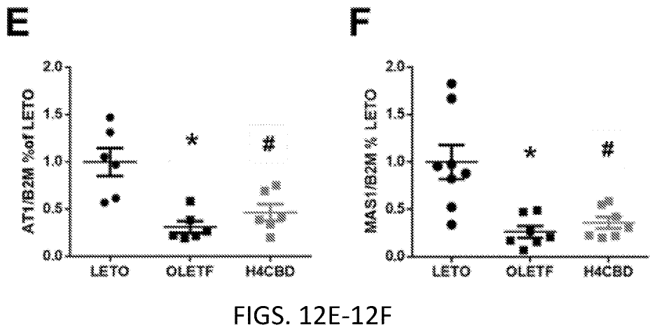

Effects of H4CBD on blood pressure and RAAS—Systolic blood pressure was monitored throughout the study via surgically implanted DSI radiotelemeters [HD-S10] to assess the effect of synthetic CBD on severe hypertension and end of study plasma concentrations of Ang 1-7, AngII and aldosterone were measured to assess modulation of drivers of hypertension. No significant difference between treated and non-treated OLETF was detected though treated OLETF demonstrated an average 6.5 mmHg (4%) increase after the 7th day of the daily dose regimen (FIG. 12A). Plasma AngII levels were 18% lower in OLETF control compared to LETO and 24% higher in treated OLETF compared to OLETF control (FIG. 12B). Plasma Ang 1-7 levels were 29% lower in OLETF compared to LETO and 26% higher in treated OLETF compared to control (FIG. 12C). Plasma aldosterone was 11% lower in OLETF control compared to LETO and 150% higher in treated OLETF compared to OLETF control (p=0.052) (FIG. 12D). AT1 mRNA relative expression was assessed to determine the effect of synthetic CBD on RAAS tone. AT1 mRNA expression was 70% lower in OLETF control compared to LETO and 49% higher in treated OLETF compared to control OLETF (FIG. 12E). Mas1 mRNA expression, an indicator of counteractive non-classical RAAS tone, was 74% lower in OLETF control compared to LETO, which was not rescued by 4 weeks of treatment with synthetic CBD (FIG. 12F).

### DISCUSSION

The effects of CBD compounds on metabolic syndrome pathophysiology have not been described. A few studies have shown beneficial^{10,11} or null effects⁷ of CBD on isolated MetS risk factors, but these effects have not been explored in context as cluster factors. Therefore, the aim of the present study was to preclinically assess the therapeutic risks and benefits in the condition of advanced MetS most frequently observed in older populations. To this end, the present study demonstrates that a long-term, high dose treatment of H4CBD decreased BM, visceral adiposity and fasting plasma TG without induction of anorexia or muscle wasting in the condition of advanced MetS. Moreover, H4CBD treatment beneficially shifted adipocyte size and increased hepatic fatty acid uptake. Despite these benefits, H4CBD treatment did not beneficially modulate RAAS and ultimately, did not reduce arterial blood pressure. These results suggest that a long-term, high dose treatment of H4CBD is not sufficient to correct MetS-associated hypertension but may be a useful tool for the activation of lipolysis to reduce obesity in conditions of severely poor glycemic state.

Synthetic CBD reduced BM despite increase in food consumption-BM and food intake were consistently higher in OLETF compared to LETO as expected^{7,8}. CBD has not been previously shown to reduce body mass de novo but rather inhibit body mass gain in rats without underlying metabolic dysfunction¹¹. The results indicate that synthetic CBD dosed daily for 1-week reduced OLETF BM to LETO control, which maintained for the duration of the study. Although relative food consumption was reduced in the treated group in the first week, by day 10, relative food consumption was higher than OLETF and LETO controls. High doses of CBD administered clinically have been noted to have anorexic and diarrheal effects indicative of gastric upset¹², which could account for the reductions noted in the first week. Daily dose was relaxed to every other day after the first week which may have offset the anorexic effects of the terpene-rich (noxious) H4CBD synthetic, indicated by the increase in relative food consumption. However, all treated animals were observed to have soft stool at multiple points of the dosing window regardless of dose frequency relaxation. BM loss was not offset by increased food consumption, which indicated H4CBD did not exert anorexic effects when dosed every other day. Therefore, the energy balance (in/out) was thought to be enhanced through increased activity, which likely accounted for the loss in BM as well as non-significant increases in 3MH/Cr. Indeed, activity scores were nearly doubled in treated OLETFs. There was an observed trend in increased energy expenditure, rather than caloric restriction, which is likely a significant contributing factor to BM loss overall.

H4CBD treatment did not reduce SBP in aged OLETF, which supports that the reduction of BM and/or visceral adiposity is not sufficient to reduce arterial blood pressure. Indeed, H4CBD did not reduce AngII or aldosterone, both of which have been shown to contribute to hypertension in the absence of AT1 blockade. Nor did H4CBD affect mRNA expression of AT1. On the other hand, the modest increase in plasma AngII may indicate a partial inhibition of AngII binding at the level of the AT1, which is evidenced in ARB-treated OLETF¹². Collectively, these findings support that the therapeutic effects of H4CBD are not sufficient to affect RAAS mediators, which is likely the dominant mechanism that elevates arterial blood pressure during advanced MetS.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1 Mascal M, Hafezi N, Wang D, Hu Y, Serra G, Dallas M L et al. Synthetic, non-intoxicating 8,9-dihydrocannabidiol for the mitigation of seizures. Sci Rep 2019; 9:7778.
2 Assa-Glazer T, Gorelick J, Sela N, Nyska A, Bernstein N, Madar Z. Cannabis Extracts Affected Metabolic Syndrome Parameters in Mice Fed High-Fat/Cholesterol Diet. Cannabis Cannabinoid Res 2020; 5:202-214.
3 Weiss L, Zeira M, Reich S, Har-Noy M, Mechoulam R, Slavin S et al. Cannabidiol lowers incidence of diabetes in non-obese diabetic mice. Autoimmunity 2006; 39:143-151.
4 Ishida K, Mizuno A, Min Z, Sano T, Shima K. Which is the primary etiologic event in Otsuka Long-Evans Tokushima Fatty rats, a model of spontaneous non-insulin-dependent diabetes mellitus, insulin resistance, or impaired insulin secretion? Metabolism 1995; 44:940-945.
5 Ewing L E, Skinner C M, Quick C M, Kennon-McGill S, McGill M R, Walker L A et al. Hepatotoxicity of a Cannabidiol-Rich Cannabis Extract in the Mouse Model. Molecules 2019; 24:1-17.
6 Mascal M, Hafezi N, Wang D, Hu Y, Serra G, Dallas M L, et al. Synthetic, non-intoxicating 8,9-dihydrocannabidiol for the mitigation of seizures. Sci Rep 2019; 9:7778.
7 Remiszewski P, Jarocka-Karpowicz I, Biernacki M, Jastrząb A, Schlicker E, Toczek M, et al. Chronic cannabidiol administration fails to diminish blood pressure in rats with primary and secondary hypertension despite its effects on cardiac and plasma endocannabinoid system, oxidative stress and lipid metabolism. Int J Mol Sci 2020; 21:1-24.
8 Kawano K, Hirashima T, Mori S, Natori T. OLETF (Otsuka Long-Evans Tokushima Fatty) rat: a new NIDDM rat strain. Diabetes Res Clin Pract 1994; 24 Suppl: S317-20.
9 Kawano K, Hirashima T, Mori S, Saitoh Y, Kurosumi M, Natori T. Spontaneous long-term hyperglycemic rat with diabetic complications: Otsuka Long-Evans Tokushima Fatty (OLETF) strain. Diabetes 1992; 41:1422-8.
10 Ewing L E, Skinner CM, Quick C M, Kennon-McGill S, McGill M R, Walker L A, et al. Hepatotoxicity of a Cannabidiol-Rich Cannabis Extract in the Mouse Model. Molecules 2019; 24:1-17.
10 Rajesh M, Mukhopadhyay P, Btkai S, Patel V, Saito K, Matsumoto S, et al. Cannabidiol attenuates cardiac dysfunction, oxidative stress, fibrosis, and inflammatory and cell death signaling pathways in diabetic cardiomyopathy. J Am Coll Cardiol 2010; 56:2115-25.
11 Ignatowska-Jankowska B, Jankowski M M, Swiergiel A H. Cannabidiol decreases body weight gain in rats: Involvement of CB2 receptors. Neurosci Lett 2011; 490:82-4.
12 Huestis M A, Solimini R, Pichini S, Pacifici R, Carlier J, Busardò F P. Cannabidiol Adverse Effects and Toxicity. Curr Neuropharmacol 2019; 17:974-89.
13 Thorwald M A, Godoy-Lugo J A, Rodriguez G J, Rodriguez M A, Jamal M, Kinoshita H, et al. Nrf2-related gene expression is impaired during a glucose challenge in type II diabetic rat hearts. Free Radic Biol Med 2019; 130:306-17.

What is claimed is:

1. A method of treating metabolic dysfunction or a condition associated with metabolic dysfunction comprising administering a therapeutically effective amount of a composition comprising H4CBD to an individual in need thereof.

2. The method of claim 1, wherein the condition associated with metabolic dysfunction is selected from obesity, insulin resistance, reduced glucose tolerance, and metabolic syndrome.

3. The method of claim 1, wherein the individual has been diagnosed with metabolic dysfunction or a condition associated with metabolic dysfunction.

4. The method of claim 1, wherein the method decreases body mass.

5. The method of claim 4, wherein the decrease in body mass comprises a reduction in abdominal fat.

6. The method of claim 1, wherein the method increases insulin receptor expression.

7. The method of claim 1, wherein the method reduces circulating adiponectin.

8. The method of claim 1, wherein the method increases circulating ghrelin.

9. The method of claim 1, wherein the method reduces plasma triglycerides.

10. The method of claim 1, wherein the method increases lipid metabolism.

11. The method of claim 1, wherein the H4CBD is a pharmaceutically acceptable salt, enantiomer, diastereomer, or prodrug thereof.

12. The method of claim 1, wherein the composition is administered orally, intraadiposally, intraarterially, intraarticularly, intracranially, intradermally, intralesionally, intramuscularly, intraperitoneally, intrapleurally, intranasally, intraocularally, intrapericardially, intraprostaticaly, intrarectally, intrathecally, intratumorally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

13. The method of claim 1, wherein the composition is administered to the subject at least two, three, four, five, six, seven, eight, nine or ten times.

14. The method of claim 1, wherein the H4CBD is administered at a dosage of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, or 20 mg/kg.

15. A method of reducing body mass in an individual comprising administering to the individual a therapeutically effective amount of a composition comprising H4CBD.

16. The method of claim 15, wherein the reduced body mass comprises a reduction in abdominal fat.

17. The method of claim 15, wherein the method increases insulin receptor expression.

18. The method of claim 15, wherein the method reduces circulating adiponectin.

19. The method of claim 15, wherein the method increases circulating ghrelin.

20. The method of claim 15, wherein the method reduces plasma triglycerides.

21. The method of claim 15, wherein the method increases lipid metabolism.

22. The method of claim 15, wherein the H4CBD is a pharmaceutically acceptable salt, enantiomer, diastereomer, or prodrug thereof.

23. The method of claim 15, wherein the composition is administered orally, intraadiposally, intraarterially, intraarticularly, intracranially, intradermally, intralesionally, intramuscularly, intraperitoneally, intrapleurally, intranasally, intraocularally, intrapericardially, intraprostaticaly, intrarectally, intrathecally, intratumorally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

24. The method of claim 15, wherein the composition is administered to the subject at least two, three, four, five, six, seven, eight, nine or ten times.

25. The method of claim 15, wherein the H4CBD is administered at a dosage of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, or 20 mg/kg.


SEQUENCE LISTING (2 sequences)

SEQ ID NO: 1 - DNA, 20 bp, synthetic construct
  source 1..20: mol_type = other DNA
aacacatgggcctcccattc

SEQ ID NO: 2 - DNA, 20 bp, synthetic construct
  source 1..20: mol_type = other DNA
aacaggtagaggacccgcat